SEQUENCE LISTING (3 sequences)

SEQ ID NO: 1 - DNA, 21 bp, Mus musculus
gctcttcctctctgtgtcctg

SEQ ID NO: 2 - DNA, 24 bp, Mus musculus
cgaatgcatagaattcagatagcc

SEQ ID NO: 3 - DNA, 22 bp, Escherichia coli
gcctgcttgccgattatcatgg

(19) United States
(12) Patent Application Publication
(10) Pub. No.: US 2008/0295192 A1
(43) Pub. Date: Nov. 27, 2008

(54) SELF-INDUCED DELETION OF DNA

(75) Inventors: Kirk R. Thomas, Salt Lake City, UT (US); Kenneth E. Bernstein, Atlanta, GA (US); Michaeline Bunting, Salt Lake City, UT (US); Joy Greer, Salt Lake City, UT (US); Mario Capecchi, Salt Lake City, UT (US)

Correspondence Address: ROTHWELL, FIGG, ERNST & MANBECK, P.C., 1425 K STREET, N.W., SUITE 800, WASHINGTON, DC, 20005, US

(73) Assignee: UNIVERSITY OF UTAH, Salt Lake City, UT (US)

(21) Appl. No.: 11/759,574

(22) Filed: Jun. 7, 2007

Related U.S. Application Data
(63) Continuation of application No. 09/606,222, filed on Jun. 29, 2000, now abandoned.
(60) Provisional application No. 60/141,267, filed on Jun. 30, 1999.

(51) Int. Cl. A01K 67/027; C07H 21/04; C12N 15/63

(52) U.S. Cl. 800/18; 536/23.1; 435/440

(57) ABSTRACT

The present invention is directed to a method for deleting DNA sequences in a tissue specific manner. In one embodiment, DNA sequences are specifically deleted in germline tissue. In a second embodiment, DNA sequences are specifically deleted in desired somatic tissue. The present invention is further directed to a nucleic acid molecule for use in the method. More specifically, a nucleic acid molecule is provide by the present invention which comprises (a) a recombinase site, (b) a tissue-specific promoter, (c) a recombinase gene, (d) a foreign DNA, and (e) a recombinase site. The nucleic acid molecule may further comprise a gene which is desired to be incorporated into and expressed in a transgenic organism. The method can be used in both plants and animals, and has many applications as described herein.

### CROSS-REFERENCE TO RELATED APPLICATION

The present application is a continuation of U.S. patent application Ser. No. 09/606,222 filed on 29 Jun. 2000, which in turn is related and claims priority under 35 U.S.C. § 119(e) to U.S. provisional patent application Ser. No. 60/141,267 filed on 30 Jun. 1999, each incorporated herein by reference.

This application was made with Government support from National Institutes of Health under Grant Nos. DK49219 and R01 DK51445. The federal government has certain rights in this invention.

### BACKGROUND OF THE INVENTION

The present invention is directed to a method for deleting nucleic acid sequences in a tissue specific manner. The present invention is further directed to a DNA molecule for use in the method.

The publications and other materials used herein to illuminate the background of the invention or provide additional details respecting the practice are incorporated by reference, and for convenience are respectively grouped in the appended List of References.

The paradigm for targeted germ-line modification of a mammalian genome was established twelve years ago (Thomas et al., 1987). An alteration introduced in vitro into a cloned gene is transferred by homologous recombination to its chromosomal target in a pluripotent embryo-derived stem (ES) cell. Cells containing the modification are placed in an embryonic environment, allowed to grow, differentiate, and to contribute to the germline of the host organism. Limitations imposed by the transformation and recombination efficiencies of mammalian cells require that the alteration of interest be linked physically to a selectable genetic marker, typically a gene encoding drug resistance under transcriptional control of a constitutive promoter/enhancer element. This operational requirement can have unpredictable consequences in vivo, such as misregulation of adjacent genes or the attenuation of expression of the gene of interest (Olson et al, 1996). Thus, the elimination of the marker may be desirable, and for technical reasons is generally performed through use of site-specific recombinase systems such as Cre/loxP (Sternberg et al., 1981) or FLP/FRT (Broach et al., 1980).

Although the prior art has developed means to remove the marker gene, it is desired to improve upon such means and to provide for better control of the process. These objects are accomplished by the present invention.

### SUMMARY OF THE INVENTION

The present invention is directed to a method for deleting nucleic acid sequences in a tissue specific manner. In one embodiment, nucleic acid sequences are specifically deleted in germline tissue. In a second embodiment, nucleic acid sequences are specifically deleted in desired somatic tissue. The present invention is further directed to a DNBA molecule for use in the method.

More specifically, a method is provided by the present invention for the self-excision of nucleic acid sequences in a tissue specific manner. According to this method, a promoter specific to a given tissue, is used to drive expression of the Cre or FLP recombinase. In one embodiment, a gamete-specific promoter, such as a testes-specific promoter or an ovary-specific promoter is used to drive expression of the Cre or FLP recombinase. In this embodiment, foreign DNA, such as a marker gene, linked to Cre or FLP, survives selection in cultured cells and remains integrated in somatic cells, but is removed along with the Cre or FLP as both are passed through the germline. In a second embodiment, a somatic tissue specific promoter, such as a muscle specific promoter, is used to drive expression of the Cre or FLP recombinase. In this embodiment, foreign DNA which is integrated in somatic cells is removed along with the Cre or FLP in the specific tissue under control of the tissue specific promoter. The method can be used in both plants and animals and has many applications as described herein.

More specifically, a DNA acid molecule is provided by the present invention which comprises (a) a recombinase site, (b) a tissue-specific promoter, (c) a recombinase gene, (d) a foreign DNA and (e) a recombinase site. In one embodiment, the tissue specific promoter is a gamete-specific promoter. In a second embodiment, the tissue specific promoter is a somatic tissue specific promoter. The DNA molecule may further comprise a gene which is desired to be incorporated into and expressed in an organism, including a transgenic organism.

A transgenic organism containing the nucleic acid molecule is further provided by the present invention.

### BRIEF DESCRIPTION OF THE FIGURES

FIG. 1 shows testes-specific self-excision. In FIG. 1A, a selectable marker gene Neo^{r}, with a constitutive promoter, is transferred by homologous recombination to a specific locus in a mouse ES cell. The Neo^{r} gene is linked to a Cre gene that is under transcriptional control of the tACE promoter, and the two genes are flanked with loxP sites (P); the entire cassette, ACN, is introduced by gene targeting to a specific locus in a mouse ES cell. In FIG. 1B, ES cells, heterozygous for an allele containing the integrated cassette, are injected into wild-type mouse blastocysts and the blastocysts allowed to develop; the resulting animals are chimeric for wild-type (host-derived) cells (white) and ES-derived cells (black). As shown in FIG. 1C, male chimeric animals will transmit through their sperm one of two alleles of the locus of interest: wild-type (white) or mutant (gray); after self-excision has occurred, the mutant allele will be marked only by a loxP site, the final product of the testes-specific self-excision reaction.

FIG. 2 shows targeting of a self-excision cassette to Hoxa3. In FIG. 2A is shown the self-excision cassette, ACN. Testes-specific elements from the mouse ACE gene (black arrow) are placed 5′ of the modified Cre structural gene (Gu et al., 1992) (red), followed, 3′, with the minimal polyadenylation signal from HSV-TK (Thomas et al., 1987) (white box). An intron, derived from the SC40 t-antigen gene (white box) is inserted into the Cre gene, the Neo^{r} gene (blue) is controlled by a promoter from the mouse RNA polymerase II gene (black arrow) and followed also by the HSV-TK poly(A) sit (white box). The 5′ and 3′ ends of this cassette contain loxP sites (P). FIG. 2B shows gene targeting at Hoxa3. In the top line, the targeting vector pRVa3^{ACN} contains 11 kb of mouse genomic DNA into which the self-excision cassette ACN has been inserted in the homeodomain of Hoxa3 (McGinnis et al., 1984), the genomic sequences are linked to the HSV-TK gene (dark gray) and are all contained on a pUC-based plasmid backbone (light gray). The ACN cassette contains at its 5′ end an Sst1 site (S), used as a marker for homologous integration of the cassette at the Hoxa3 gene. On the second line, the wild-type Hoxa3 locus, and the bottom line, the predicted structure of the recombinant Hoxa3^{ACN} allele. The 5′ flanking probe used to detect recombination is indicated, and the diagnostic SstI-generated DNA fragments delineated beneath each locus. Yellow boxes designate Hoxa3 exons, other SstI sites in the vector are not indicated. In FIG. 2C, in Southern transfer analysis, DNA from the parental cell line (ES) and the homologous recombinant ES lines used to generate mice was restricted with SstI. Radiolabelled DNA probe is depicted in b.

FIG. 3 shows genetic transmission of Hoxa3 alleles. In FIG. 3A, the PCR-based genotyping of the three Hoxa3 alleles shows primer 1 (p1) is from the Hoxa3 intron, primer 2 (p2) is from coding exon 2-derived sequences (antisense), and primer 3 (p3) is from the Neo^{r} gene. Predicted sites are indicated, color coding is as in FIG. 2. FIG. 3B shows genotyping of DNA from wild-type ES cells (ES), recombinant ES cell line, 1h-9, tail biopsies from a chimeric male, χ3227, generated from 1h-9, and tail tissue from F₁ progeny of the chimera. Amplified DNA was electrophoresed through agarose and stained with ethidium bromide. Sizes correspond to those listed in FIG. 3A. FIG. 3C shows the absence of excision in somatic tissue. A single chimeric male derived from cell line 1h-9 was sacrificed at eight weeks of age. DNA extracted from each of the indicated tissues was analyzed by PCR as in FIG. 3B. Analysis of a second chimera showed an identical result.

### DETAILED DESCRIPTION OF THE INVENTION

In accordance with a first aspect of the present invention, a method is provided for the self-excision of nucleic acid sequences in desired tissues of organisms, i.e., plants or animals. According to this aspect, a DNA molecule, as described herein, which has been designed to provide deletion of a foreign DNA in the desired tissue of an organism is introduced into an organism. The organism is grown resulting in the excision of the foreign DNA in the desired tissue. In one embodiment, the DNA molecule is introduced to produce a transgenic organism. Alternatively, the nucleic acid molecule could be introduce into an organism, such as in gene therapy. In one embodiment, the method provides for the self-excision of nucleic acid sequences in the germline. In this embodiment, the foreign DNA is excised in the transgenic organism during gametogenesis. In a second embodiment, the method provides for the self-excision of nucleic acid sequences in specific tissue, In this embodiment, the foreign DNA is excised in the specific somatic tissue during growth of the organism. The “foreign” DNA may be heterologous DNA, such as a marker sequence, or it may be a wild-type allele, such as for use in gene therapy, and its presence in the germline of the transgenic organism or in certain tissue of the organism is usually not desired. The DNA molecule may further contain a gene which is desired to be incorporated into the transgenic organism or into tissue in the organism. The method of the present invention prevents germline transmission of the foreign DNA or prevents somatic expression of the foreign DNA in non-desired tissue.

In accordance with a second aspect of the present invention, a DNA molecule is provided which is useful in the method of the present invention. The DNA molecule comprises (a) a recombinase site, (b) a tissue specific promoter, (c) a recombinase gene, (d) a foreign DNA and (e) a recombinase site. In one embodiment, the tissue specific promoter is a gamete-specific promoter. In a second embodiment, the tissue specific promoter is a somatic tissue-specific promoter. The DNA molecule may further comprise a gene which is desired to be incorporated into and expressed in an organism. The foreign DNA may be heterologous DNA, such as a marker sequence, or it may be a wild-type allele, such as for use in gene therapy, and its presence in the germline of the transgenic organism is usually not desired. Examples of recombinase sites include, but are not limited to, loxP and FRT. Examples of recombinase genes include, but are not limited to, Cre and FLP. The foreign DNA survives preparing transgenic cells, selection of transgenic cells, and in somatic cells remains integrated but (a) in one embodiment is excised during gametogenesis as the transgenic organism grows or (b) in a second embodiment is excised in a tissue specific manner as the transgenic organism grows.

The present invention is further described with reference to a first embodiment in which nucleic acid sequences are deleted as they pass through the germiline of plants or animals. It is understood that the method is also applicable to deletion of nucleic acid sequences in specific tissues of plants or animals through the use of a particular tissue specific promoter in place of the gamete-specific promoter discussed in this description.

A procedure is described that directs self-induced deletion of nucleic acid sequences as they pass through the germline of plants or animals. Although the method of the present invention is illustrated with reference to male germline of animals and using Cre, it is to be understood that the method is also applicable to female germline of animals, male germline of plants and female germline of plants and the use of other recombinase systems. As detailed herein, the testes-specific promoter from the angiotensin-converting enzyme gene is used to drive expression of the Cre-recombinase gene. Cre was linked to the selectable marker, Neo^{r}, and the two genes flanked with loxP elements. This cassette was targeted to the Hoxa3 gene in mouse ES cells that were in turn used to generate chimeric mice. In these chimeras, somatic cells derived from the ES cells retained the cassette, but self-excision of the marker gene was found to have occurred in all ES-cell-derived sperm.

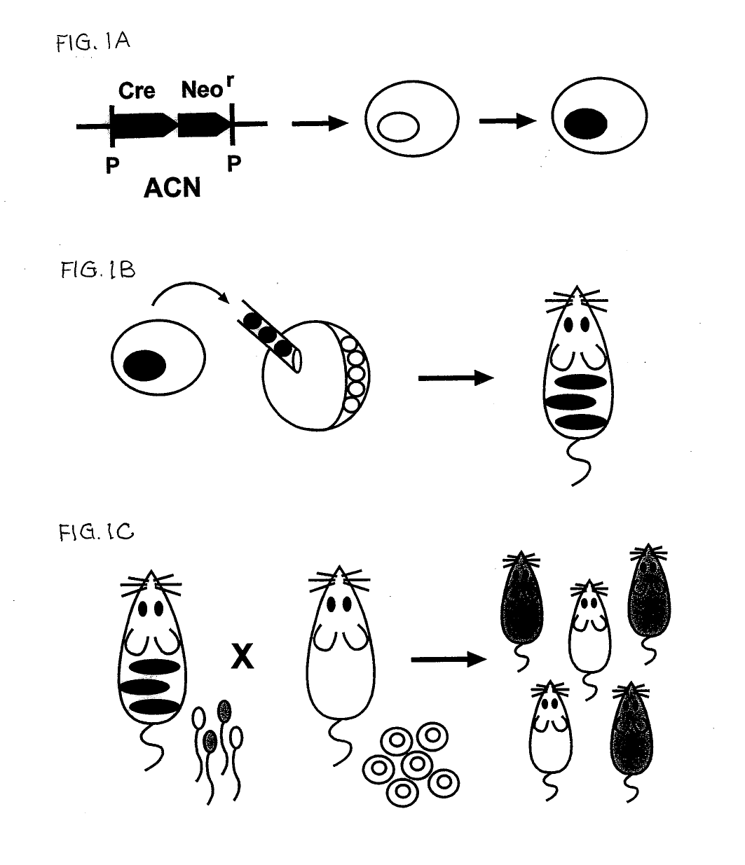

The strategy behind the present invention protocol is illustrated in FIG. 1: the intragenic promoter of the murine angiotensin converting enzyme, tACE (shown to initiate transcription only during spermatogenesis), directs expression of Cre; tACE-Cre is linked to the selectable marker gene, Neo^{r}, and the two genes, tACE-Cre/Neo^{r}, are flanked with loxP sites. This cassette, referred to as ACN, is targeted by homologous recombination to a specific locus in a murine ES cell. Cells containing the appropriate chromosomal recombinant are inserted into a blastocyst-stage mouse embryo which develops into a chimeric animal, containing cells from both the host blastocyst and the cassette-containing ES cells. If the chimerism extends to the germline of an adult male, some fraction of the sperm will be ES-cell derived. During spermatogenesis the tACE promoter induces expression of the Cre-recombinase, the ACN cassette is excised, and a single loxP element remains at the chromosomal locus. Progeny from these sperm should represent two classes of paternal transmission: (1) those containing a wild-type paternal chromosome, originating either from the non-targeted chromosome in the heterozygous ES cells or from non-ES (i.e. host)-derived cells; and (2) those containing a loxP insertion in the paternal chromosome.

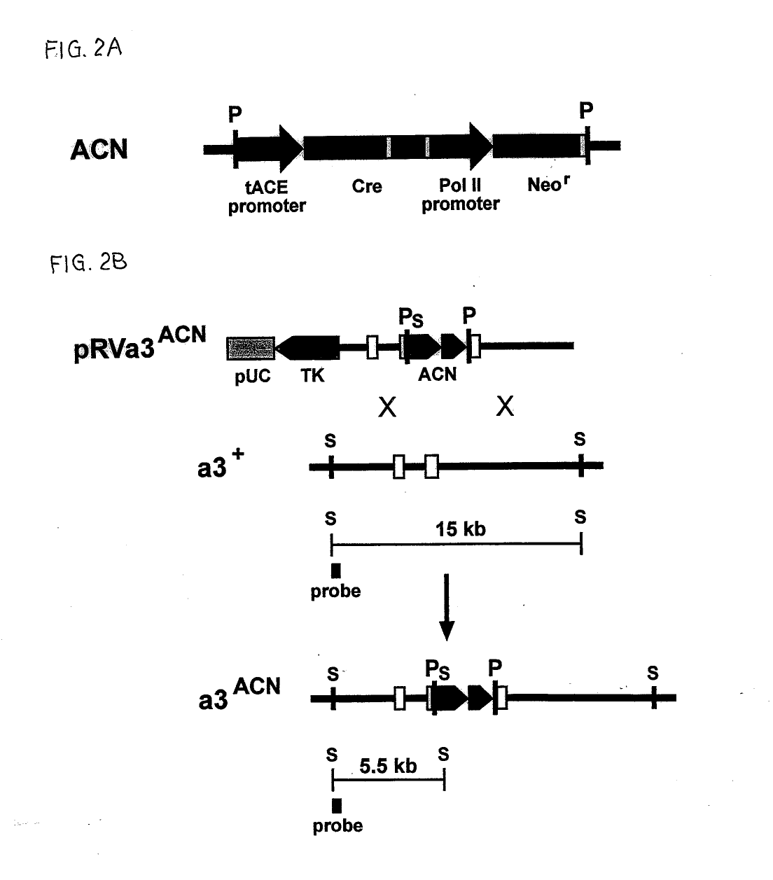

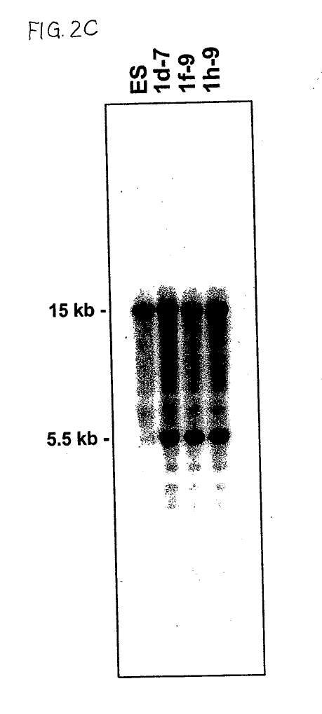

The experimental design used to test this protocol is illustrated in FIG. 2. Two features of the ACN-cassette should be noted: Neo^{r} is located 3′ of the tACE-Cre gene, such that transcription of Neo^{r} should not result in transcriptional read-through of Cre; and the Cre gene contains an intron to prevent in-frame translation and subsequent self-excision in bacteria. We inserted the ACN cassette into a genomic clone of the mouse Hoxa3 gene, and transfected the targeting vector into mouse ES cells. We clonally isolated 144 cell lines that survived positive-negative selection, and demonstrated by Southern transfer analysis that 20 contained the ACN-cassette integrated into one of the endogenous Hoxa3 loci.

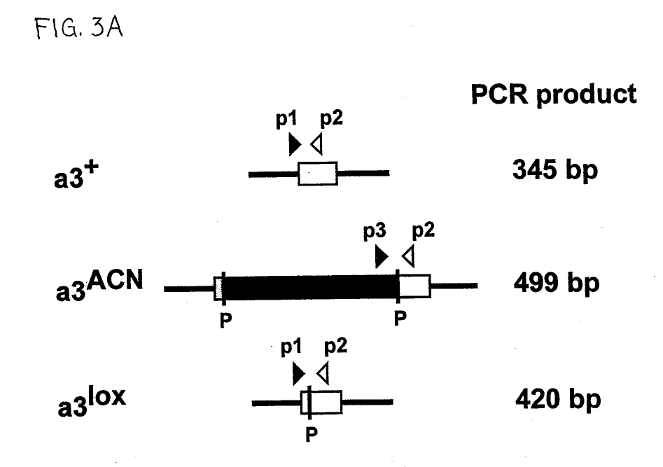

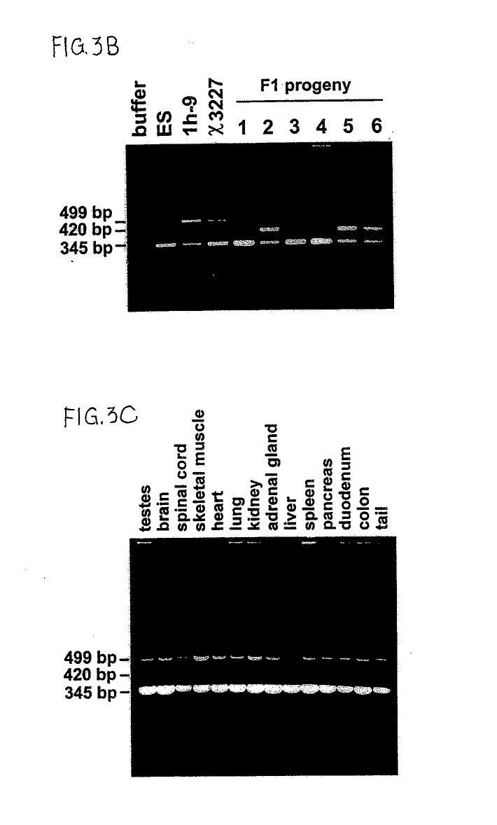

Three of the recombinant ES-cell lines were used to generate 13 male chimeric mice that in turn sired 138 ES-cell-derived progeny (determined by coat color). All progeny were genotyped by a PCR-based assay that could distinguish between the three potential Hoxa3 alleles: wild type, ACN, and loxP (FIG. 3A) FIG. 3B shows such an assay, comparing DNA isolated from the parental ES cell line, one recombinant ES cell line, tail biopsies from a chimeric male, and 6 of his agouti progeny. The recombinant ES cells and the chimera-derived tails are heterozygous for the wild-type and ACN-containing alleles whereas the F₁ progeny are either wild-type or heterozygous for the loxP allele. A summary of the genotypes of all 138 progeny, shown in Table 1, demonstrates that tACE-Cre mediated germline excision of the ACN cassette in all cases.

TABLE 1
Genotypic Analysis of Progeny

| Cell | No. of | Genotype of Progeny | | |
|---|---|---|---|---|
| Line | Chimeras | +/+ | +/ACN | +/lox |
| 1d-7 | 3 | 23 | 0 | 26 |
| 1h-9 | 9 | 37 | 0 | 32 |
| 1f-9 | 1 | 67 | 0 | 13 |
| Total | 13 | 67 | 0 | 71 |

Male chimeric animals derived from 3 cell lines were mated with C57B1/6 females. DNA was extracted from tails of all agouti pups and was genotyped as described herein. The number of animals with each genotype is indicated.

Although self-excision was complete at the level of spermatogenesis, it was also restricted to the testes. Tissues from chimeric males that transmitted the loxP allele were genotyped, and with the exception of the testes were heterozygous for the wild-type and the ACN alleles (FIG. 3C). Testes, which were mosaic for the two mutant Hoxa3 alleles, include multiple cell types, only two of which, the elongating spermatids and the spermatozoa, should contain the loxP allele.

A similar protocol has been used to generate mice carrying a loxP insertion in the Hoxd3 gene, which demonstrates the applicability of the present invention to a vast number of loci. The tACE promoter is inactive in somatic cells when integrated at independent ectopic sites. It also appears refractory to activation when integrated at random loci in ES cells, even when linked to a transcriptionally active Neo^{r} gene. Were the tACE promoter frequently expressed following integration in ES cells, the capacity of DNA carrying the self-excision cassette to generate stable transformants would be greatly reduced, but this is not the case. It remains possible, however, that if the cassette were targeted to a transcriptionally active locus, that the Cre protein could be translated from read-through mRNA transcribed into the cassette. Under such conditions, it would be necessary to custom-design a cassette containing transcription insulators or to place ACN in a transcriptional orientation opposite to that of the target locus.

The present method has many applications with plants and animals. One application is in the generation of knockout animals. The possibility that a marker gene may unpredictably affect phenotype has already prompted removal of such sequences prior to phenotypic analysis. Although alternative recombinase-based excision methods do exist, they are often accompanied with operational inconveniences. For example, removal of sequences during the growth of ES cells requires additional selection and/or screening. Not only is there a time and labor consideration involved in such manipulation, but the pluri-potency of ES cells can be adversely affected by prolonged growth in culture. Sequence deletion in the animal relies either on the expression of the recombinase in the fertilized eggs of animals carrying a loxP-flanked gene, the mating of such animals with a Cre-expressing mouse, or the use of ES cells containing a Cre-expressing transgene. All methods require additional breeding and/or technical expertise, and thus prolong by several months the time required for analysis.

The above pragmatic advantage will also be realized in the generation of chromosomal rearrangements typically mediated by Cre-catalyzed recombination or in the condensation of tandem repeats resulting from the random integration of transgenes following pronuclear injection. Linkage of tACE-Cre to a loxP site defining the desired deletion endpoint should greatly simplify these chromosomal engineering processes.

A further application of the present invention is the generation of mice harboring conditional-mutant alleles. The creation of such animals often takes advantage of either the Cre/loxP or FLP/FRT recombination systems to create genetic deletions regulated by the restricted spatial or temporal expression of the appropriate recombinase. The recombinogenic elements, loxP or FRT, must first be introduced into the genome by linkage to a selectable marker gene. Because it is essential that the ground state of such experiments be wild-type, it is imperative that the marker gene not influence the expression of the target gene. If the two recombinase systems were employed in the same animal, for example, the self-excising cassette expressing FLP, and deletion elements responding to the conditional expression of Cre, such a requirement could be met.

Another application of the self-excision method of the present invention is in the area of agricultural crops. New strains of agricultural crops are now equipped with ‘terminator’ genes to limit the propagation of proprietary traits. A self-excision mechanism activated only in the germline would provide a single step method to restrict those traits to a single, founding generation, and may reduce the threat of unintended transmission of genetic traits to non-target species.

In addition, the present method can be used as part of in utero human gene therapy as a means to correct genetic deficiencies. Because such protocols will induce genetic changes in embryonic cells, including those that may colonize the germline, they have raised both moral and pragmatic objections. If, however, such modifications were linked with a germline-expressed recombinase and flanked with recombinogenic elements, the challenges to such modifications will be removed along with the intervening DNA.

The present method can also be used to delete undesired DNA, such as may be introduced in gene therapy, in a tissue in which expression is not desired.

### EXAMPLES

The present invention is further detailed in the following Examples, which are offered by way of illustration and are not intended to limit the invention in any manner. Standard techniques well known in the art or the techniques specifically described below are utilized. GenBank accession numbers: SV40 t-antigen (J02400); loxP (M10287); RNA polymerase II large subunit (M14101); ACE (M61094); Neo (V00618).

### Example 1

### Vector Construction

The self-excision cassette was assembled into the bacterial plasmid, pBS (Stratagene) using standard cloning methods. The tACE promoter sequences are nucleotides 495 to 1194; the Cre gene includes the entire protein-coding domain from pMC1-Cre followed by the minimal polyA sequence from the HSV-TK gene; intron sequences from the SV40 t-antigen gene, nucleotides 4637-4572, were amplified by PCR and inserted between codons 283 and 284 of Cre; the Neo^{r} gene is an 873-bp PstI to BamHI fragment isolated from pMC1Neo-polyA; the promoter includes bases 1 to 713 from the mouse RNA polymerase II large subunit gene; the 34-bp minimal loxP sites are in parallel orientation at each end of the cassette. Murine Hoxa3 sequences were isolated from a λ phage library constructed in this laboratory of genomic DNA isolated from ES cells. Sequences used for the targeting vector extend from a Sau3A1 site, 2.2 kb upstream of the ATG in exon 1 to an EcoRI site 5.5 kb 3′ of the TGA in exon 2. ACN was inserted into the BglII site in the homeodomain in exon2. An 8-bp ExoRV-containing oligonucleotide linker was also inserted at the Eco47III-site in exon 1. This introduces a premature stop codon, creating an allele of Hoxa3 to be used in future studies of this locus.

### Example 2

### ES Cells

### Transformation, Screening and Blastocyst Injection

The targeting vector, pRVa3^{ACN}, was introduced in linear form by electroporation into RI ES cells that were subsequently selected for resistance to G418 and FIAU. Approximately 2×10⁷ cells were subjected to electroporation and 144 drug-resistant colonies isolated. DNA was extracted from cells of each clone and subjected to analysis by Southern transfer under previously described conditions. Homologous recombination was verified following digestion with two separate restriction endonucleases and hybridization with three individual probes. No rearrangements other than the predicted homologous recombination reaction were seen, nor were any homologous recombination events accompanied by detectable random integration of vector sequences. Cells from clones identified as heterozygous at the Hoxa3 locus were injected into C57B1/6-derived blastocysts that were allowed to come to term. Chimeric progeny were identified by coat color and those males estimated to contain >80% ES cell contribution were mated with C57B1/6 females.

### Example 3

Tissue and Cell Genotype Analysis

DNA was extracted from tail biopsies of chimeric males and their progeny, as well as from tissues isolated from euthanized chimeric animals, and resuspended in TE buffer. Approximately 1 μg of DNA was dissolved in 40 μl of a PCR lysis buffer, denatured at 95° C. for five minutes, and quick-chilled on ice. Five microliters of the denature DNA solution was amplified for 30 cycles in a 25-μl reaction mixture under previously described reaction conditions and cycling parameters. Primer sequences were as follows: Primer 1: 5′-GCTCTTCCTCTCTGTGTCCTG-3′ (SEQ ID NO: 1), represents sequences 5′ of the splice acceptor site in the Hoxa3 intron; Primer 2: 5′-CGAATGCATAGAATTCAGATAGCC-3′ (SEQ ID NO:2), is antisense sequence from Hoxa3, nucleotides 849 to 826; primer 3: 5′-GCCTGCTTGCCGATTATCATGG-3′ (SEQ ID NO:3), is from the sense strand of the Neo^{r} gene, nucleotides 2121 to 2142. Amplified products were analyzed by electrophoresis through 3% NuSieve 3:1 agarose (FMC). FIG. 3B shows products from single, 25 μl reactions; FIG. 3C contains pools of eight amplification reactions.

While the invention has been disclosed in this patent application by reference to the details of preferred embodiments of the invention, it is to be understood that the disclosure is intended in an illustrative rather than in a limiting sense, as it is contemplated that modifications will readily occur to those skilled in the art, within the spirit of the invention and the scope of the appended claims.

### LIST OF REFERENCES

Araki, K. M., et al. (1995). Site-specific recombination of a transgene in fertilized eggs by transient expression of Cre-recombinase. Proc. Natl. Acad. Sci. USA 92: 160-164.
Broach, J. R., et al. (1980). Replication and recombination functions associated with the yeast plasmid, 2m circle. Cell 21:501-508.
Capecchi, M. R. (1999). Human germline gene therapy: a discussion on how and why. In: Engineering the Human Germline (ed. G. Stock, J. Campbell), Oxford Univ. Press, New York, N.Y.
Colledge, W. H., et al. (1995). Generation and characterization of a DF508 cystic fibrosis mouse model, Nature Genet. 10:445-452.
Deng, C., et al. (1993). Location of crossovers during gene targeting with insertion and replacement vectors, Mol. Cell. Biol. 13:2134-2140.
Ewbank, J. (1998). Problems of germline therapy, Nature 392: 645.
Garrick, D., et al. (1998). Repeat-induced gene silencing in mammals, Nature Genet. 18: 56-59.
Gu, H., et al. (1994). Deletion of a DNA polymerase beta gene segment in T cells using cell type-specific gene targeting, Science 265:103-106.
Gu, H., et al. (1993). Independent control of immunoglobulin switch recombination at individual switch regions evidenced through Cre-loxP-mediated gene targeting, Cell 73:1155-1164.
Hasty, P., et al. (1991). Introduction of a subtle mutation into the Hox-2.6 locus in embryonic stem cells, Nature 350:243-246.
Hérault, Y., et al. (1998). Engineering chromosomes in mice through targeted meiotic recombination (TAMERE), Nature Genet. 20:381-384.
Langford, K. G., et al. (1991). Transgenic mice demonstrate a testis-specific promoter for angiotensin-converting enzyme, J. Biol. Chem. 266:15559-15562.
McGinnis, W., et al. (1984). Molecular cloning and chromosome mapping of a mouse DNA sequence homologous to homeotic genes of Drosophila, Cell 38:675-680.
McLaren, A. (1998). Problems of germline therapy, Nature 392: 645.
Meyers, E. N., et al. (1998). An Fgf8 mutant allelic series generated by Cre- and Flp-mediated recombination, Nature Genet. 18:136-141.
Nagy, A., et al. (1993). Derivation of completely cell culture-derived mice from early-passage embryonic stem cells, Proc. Natl. Acad. Sci. USA 90:8424-8428.
O'Gorman, S., et al. (1997). Protamine-Cre recombinase transgenes efficiently recombine target sequences in the male germ line of mice, but not in embryonic stem cells, Proc. Natl. Acad. Sci. USA 94:14602-14607.
Olson, E. N., et al. (1996). Know your neighbors: Three phenotypes in null mutants of the myogenic bHLH gene MRF4, Cell 85: 1-4.
Pikaart, M. J., et al. (1998). Loss of transcriptional activity of a transgene is accompanied by DNA methylation and histone deacetylation and is prevented by insulators, Genes Dev. 12:2852-2862.
Ramirez-Solis, R., et al. (1995). Chromosome engineering in mice, Nature 378:720-724.
Rossant, J., et al. (1999). “Cre”-ating mouse mutants—a meeting review on conditional mouse genetics, Genes Dev. 13:142-145.
Schwenk, F., et al. (1995). A cre-transgenic mouse strain for the ubiquitous deletion of loxP-flanked gene segments including deletion in germ cells. Nucleic Acids Res. 23:5080-5081.
Service, R. F. (1998). Seed-sterilizing ‘terminator technology’ sows discord, Science 282:850-851.
Smith, A. J. H., et al. (1995). A site-directed chromosomal translocation induced in embryonic stem cells by Cre-loxP recombination, Nature Genet. 9:376-385.
Spyropoulos, D. D., et al. (1994). Targeted disruption of the even-skipped gene, evx1, causes early postimplantation lethality of the mouse conceptus, Genes Dev. 8:1949-1961.
Sternberg, N., et al. (1981). Bacteriophage P1 site-specific recombination. I. Recombination between loxP sites, J. Mol. Biol. 150:467-486.
Thomas, K. R., et al. (1987). Site-directed mutagenesis by gene targeting in mouse embryo-derived stem cells, Cell 51:503-512.
Wadman, M. (1998). US gene-therapy proposals come under fire, Nature 395:309.

What is claimed is:

1. A DNA molecule for removing a nucleic acid sequence that has been inserted into a host cell, the DNA molecule comprising, flanked by recombinase sites in a single nucleotide chain, (a) a spatially or temporally restricted promoter operably linked to (b) a recombinase gene, and (c) said nucleic acid sequence to be removed.

2. The molecule of claim 1, wherein said recombinase site is selected from the group consisting of loxP and FRT.

3. The molecule of claim 1, wherein said recombinase gene is selected from the group consisting of Cre and FLP.

4. The molecule of claim 2, wherein said recombinase gene is selected from the group consisting of Cre and FLP.

5. The molecule of claim 1, wherein said molecule further comprises a gene which is desired to be expressed in a cell.

6. The nucleic acid molecule of claim 1, wherein said nucleic acid sequence is a wild-type allele or fragment thereof of a gene.

7. A method for deleting a nucleic acid sequence from a mouse cell genome in a regulatable manner utilizing a promoter, wherein said nucleic acid sequence is part of a DNA molecule comprising, flanked by recombinase sites in a single nucleotide chain, a spatially or temporally restricted promoter operably linked to a recombinase gene and said nucleic acid sequence to be removed, the method comprising inserting said DNA molecule into the genome of said mouse cell, and growing said mouse cell such that said promoter is active, said recombinase gene is expressed in the cell and said nucleic acid sequence is deleted.

8. The method of claim 7, wherein the DNA molecule further comprises a gene which is desired to be expressed in the cell.

9. The method of claim 8, wherein said nucleic acid sequence is heterologous DNA.

10. The method of claim 8, wherein the promoter is specific to the male or female gamete.

11. The method of claim 7, wherein the mouse cell is transgenic for said DNA molecule and said nucleic acid sequence is deleted during gametogenesis in the mouse.

12. The method of claim 47, wherein said nucleic acid sequence is heterologous DNA.

13. A transgenic mouse which contains a DNA molecule comprising, flanked by recombinase sites in a single nucleotide chain, (a) a spatially or temporarally restricted promoter operably linked to (b) a recombinase gene, and (c) a nucleic acid sequence to be removed, wherein said DNA molecule has been stably integrated into the genome of said transgenic mouse.

14. The method of claim 7, wherein said nucleic acid sequence is heterologous DNA.

15. The method of claim 7, wherein said nucleic acid sequence is a wild-type allele or fragment thereof of a gene.

16. The method of claim 8, wherein said nucleic acid sequence is a wild-type allele or fragment thereof of a gene.

17. The method of claim 7 wherein the cell is part of a tissue and the promoter is a promoter specifically expressed in said tissue.

18. The method of claim 17 wherein the nucleic acid molecule further comprises a gene which is desired to be expressed in the tissue.

19. The method of claim 17, wherein said nucleic acid sequence is a wild-type allele or fragment thereof of a gene.

20. The method of claim 17, wherein said nucleic acid sequence is heterologous DNA.

21. The method of claim 17 wherein said tissue is male or female gametic tissue.

22. The molecule of claim 1 wherein said recombinase gene contains an intron.

23. The molecule of claim 22 wherein said intron is a SV40t-antigen sequence.

24. The method of claim 7 wherein said recombinase gene contains an intron.

25. The method of claim 24 wherein said intron is an SV40t-antigen sequence.

26. The transgenic mouse of claim 13 wherein said recombinase gene contains an intron.

27. The transgenic mouse of claim 26 wherein said intron is an SV40t-antigen sequence.
